(19) United States
(12) Patent Application Publication
(10) Pub. No.: US 2023/0238137 A1
(43) Pub. Date: Jul. 27, 2023

(54) INFORMATION PROCESSING APPARATUS, INFORMATION PROCESSING METHOD, AND INFORMATION PROCESSING PROGRAM

(71) Applicant: FUJIFILM CORPORATION, Tokyo (JP)

(72) Inventors: Yasuhisa KANEKO, Tokyo (JP); Nobuya KITAMURA, Tokyo (JP); Tomohide HIRAGAMI, Tokyo (JP); Yasuyuki HOSONO, Tokyo (JP); Kenji NAGAMIYA, Tokyo (JP)

(21) Appl. No.: 18/062,020

(22) Filed: Dec. 6, 2022

(30) Foreign Application Priority Data
Jan. 24, 2022 (JP) 2022-008899

(51) Int. Cl. G16H 50/20; A61B 5/00; G16H 10/60

(52) U.S. Cl. CPC G16H 50/20; A61B 5/6802; G16H 10/60

(57) ABSTRACT

An information processing apparatus includes at least one processor. The processor acquires biological information of a user which is measured by a measurement device used by the user, and derives an examination method for making a diagnosis for a body of the user as a recommended examination method based on the biological information.

### CROSS-REFERENCE TO RELATED APPLICATIONS

The present application claims priority under 35 U.S.C. § 119 to Japanese Patent Application No. 2022-008899, filed on Jan. 24, 2022. The above application is hereby expressly incorporated by reference, in its entirety, into the present application.

### BACKGROUND OF THE INVENTION

### 1. Technical Field

The disclosed technology relates to an information processing apparatus, an information processing method, and an information processing program.

### 2. Description of the Related Art

The following technology is known as technology related to the selection of an examination item for examining a disease of a patient. For example, JP2008-077603A discloses an examination item selection device comprising an onset probability calculation unit that calculates a disease onset probability of a predetermined disease based on physical information of an individual, a criterion generation unit that generates a selection criterion of an examination item for examining the predetermined disease in accordance with the calculated disease onset probability, an examination item database that stores in advance a plurality of types of examination items for examining the predetermined disease and attributes of these examination items in association with each other, and a search unit that searches the examination item database for an examination item having an attribute that satisfies the selection criterion.

### SUMMARY

In the medical document data, such as an electronic medical record, medical information indicating a symptom obtained through an interview with a patient, is recorded. The doctor formulates the examination method for making a definitive diagnosis based on the medical information recorded in the medical document data. However, there is a case in which the patient is not clearly aware of his/her symptom or cannot correctly report his/her symptom to a doctor. In such a case, there is a risk that information lacking in the accuracy is recorded in the medical document data or useful information is not recorded in the medical document data, and as a result, it is difficult to formulate an appropriate examination method.

The disclosed technology is to support the formulation of an examination method for making a diagnosis for a body of a user who is a patient.

The disclosed technology relates to an information processing apparatus comprising at least one processor. The processor is configured to acquire at least one of input information which is input to a user terminal device used by a user, or biological information which is measured for the user, and derive an examination method for making a diagnosis for a body of the user as a recommended examination method based on at least one of the acquired input information or biological information.

According to the disclosed technology, it is possible to support the formulation of the examination method for making the diagnosis for the body of the user as compared with a case in which at least one of the input information, which is input to the user terminal device used by the user, or the biological information, which is measured for the user, is not acquired.

### BRIEF DESCRIPTION OF THE DRAWINGS

Exemplary embodiments according to the technique of the present disclosure will be described in detail based on the following figures, wherein:

FIG. 1 is a diagram showing an example of a configuration of an information processing system according to an embodiment of the disclosed technology;

FIG. 2 is a diagram showing an example of medical information recorded in an electronic medical record according to the embodiment of the disclosed technology;

FIG. 3 is a diagram showing an example of a hardware configuration of an information processing apparatus according to the embodiment of the disclosed technology;

FIG. 4 is a functional block diagram showing an example of a functional configuration of the information processing apparatus according to the embodiment of the disclosed technology;

FIG. 5 is a diagram showing an example of analysis processing according to the embodiment of the disclosed technology;

FIG. 6 is a diagram showing an example of the analysis processing according to the embodiment of the disclosed technology;

FIG. 7 is a diagram showing a function of an auxiliary medical information generation model according to the embodiment of the disclosed technology;

FIG. 8 is a diagram showing an example of a training method of the auxiliary medical information generation model according to the embodiment of the disclosed technology;

FIG. 9 is a diagram showing an example of generation processing according to the embodiment of the disclosed technology;

FIG. 10 is a diagram showing a function of a recommended examination method derivation model according to the embodiment of the disclosed technology;

FIG. 11 is a diagram showing an example of derivation processing according to the embodiment of the disclosed technology;

FIG. 12 is a diagram showing an example of a display form of a recommended examination method according to the embodiment of the disclosed technology;

FIG. 13 is a flowchart showing an example of a flow of processing performed by executing an information processing program according to the embodiment of the disclosed technology;

FIG. 14 is a functional block diagram showing an example of the functional configuration of the information processing apparatus according to the embodiment of the disclosed technology;

FIG. 15 is a diagram showing an example of an examination order reception screen according to the embodiment of the disclosed technology;

FIG. 16 is a diagram showing an example of a display aspect of a comparison result by a comparison unit according to the embodiment of the disclosed technology;

FIG. 17 is a diagram showing an example of the recommended examination method and the display form of the auxiliary medical information according to the embodiment of the disclosed technology;

FIG. 18 is a diagram showing an example of the display forms of the recommended examination method and the auxiliary medical information according to the embodiment of the disclosed technology;

FIG. 19 is a flowchart showing an example of a flow of processing performed by executing the information processing program according to the embodiment of the disclosed technology;

FIG. 20 is a functional block diagram showing an example of the functional configuration of the information processing apparatus according to the embodiment of the disclosed technology;

FIG. 21 is a diagram showing a function of the recommended examination method derivation model according to the embodiment of the disclosed technology;

FIG. 22 is a diagram showing an example of integration processing according to the embodiment of the disclosed technology;

FIG. 23 is a diagram showing an example of the display form of the recommended examination method according to the embodiment of the disclosed technology;

FIG. 24 is a flowchart showing an example of a flow of processing performed by executing the information processing program according to the embodiment of the disclosed technology;

FIG. 25 is a diagram showing an example of the display form of the recommended examination method according to the embodiment of the disclosed technology; and

FIG. 26 is a functional block diagram showing an example of the functional configuration of the information processing apparatus according to the embodiment of the disclosed technology.

### DETAILED DESCRIPTION

In the following, an example of embodiments of the disclosed technology will be described with reference to the drawings. It should be noted that the same or equivalent components and portions in the drawings are represented by the same reference numerals, and the overlapping description will be omitted.

### First Embodiment

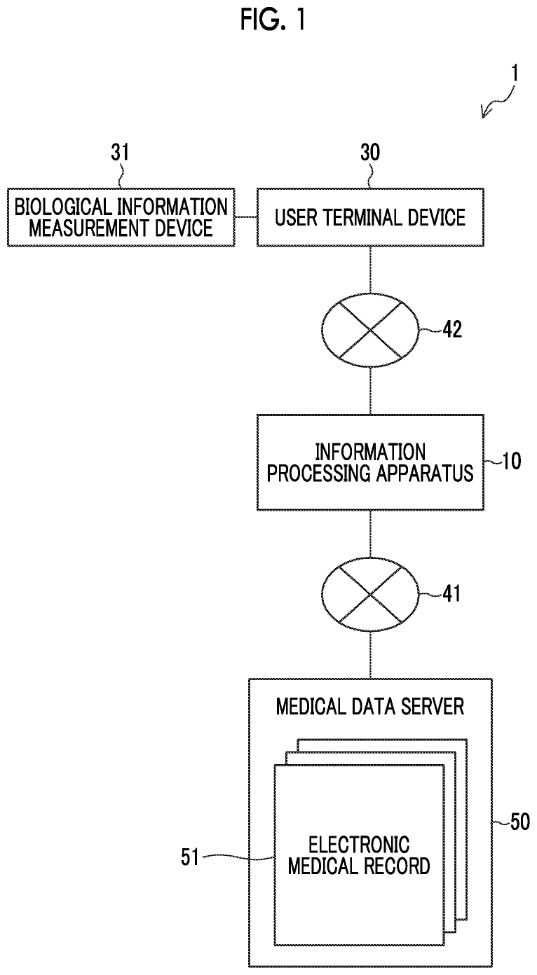

FIG. 1 is a diagram showing an example of a configuration of an information processing system 1 according to the embodiment of the disclosed technology. The information processing system 1 includes an information processing apparatus 10, a medical data server 50, a user terminal device 30, and a biological information measurement device 31.

The medical data server 50 is a computer which manages medical data of a patient. An electronic medical record 51 for each patient is stored in the medical data server 50. The electronic medical record 51 is medical document data in which medical information obtained by a medical examination of a user who is the patient is recorded.

The information processing apparatus 10 is communicably connected to the medical data server 50 via a network 41, and can access the electronic medical record 51 stored in the medical data server 50. The information processing apparatus 10 can view, create, and edit the electronic medical record 51. The information processing apparatus 10 and the medical data server 50 may be installed in a medical institution, such as a hospital, and in this case, the network 41 may be a local area network (LAN) constructed in the medical institution. Moreover, the medical data server 50 may be a so-called cloud server. In this case, the network 41 may be a wide area network (WAN).

The user terminal device 30 is a computer used by the user who is the patient of which the medical information is recorded in the electronic medical record 51. The user terminal device 30 may be, for example, a personal computer or a smartphone which is personally used by the user (patient). The user terminal device 30 can be connected to the information processing apparatus 10 via a network 42. The network 42 may be at least any one of the local area network (LAN) or the wide area network (WAN), or a combination thereof.

Various applications, such as a web browser, an e-mail application, a messenger application, and an SNS application, are installed in the user terminal device 30, and the user can use these applications. For example, the user can perform a web search by inputting a search keyword into a search engine by using the user terminal device 30. Moreover, the user can transmit or dispatch a message or information to a specified or unspecified party by using the e-mail application, the messenger application, the social networking service (SNS) application, and the like installed in the user terminal device 30. It should be noted that only one user terminal device 30 is shown in FIG. 1, but the information processing system 1 may include a plurality of user terminal devices 30.

The biological information measurement device 31 measures biological information of the user (patient). For example, the biological information may be information indicating at least one of a body temperature, a heartbeat, electrocardiography, myoelectricity, a blood pressure, arterial oxygen saturation (SpO2), a blood glucose level, or a lipid level. Moreover, the biological information may include a fluctuation rate of these measurement values in any period. The biological information measurement device 31 may be a wearable device, such as a smart watch, which comprises a sensor that measures these pieces of the biological information. The biological information measurement device 31 has a function of continuously measuring the biological information of the user in accordance with a predetermined time schedule and transmitting the measurement value to the user terminal device 30. The biological information measurement device 31 may perform measurement for various measurement items, for example, once or twice or more times daily. A measurement frequency may be different for each measurement item. Moreover, the user may be able to set any time for the time schedule of the measurement. The connection between the user terminal device 30 and the biological information measurement device 31 may be in any form of wireless or wired. Moreover, the user terminal device 30 and the biological information measurement device 31 may be connected to each other via the network configured by at least any one of the local area network (LAN) or the wide area network (WAN), or a combination thereof.

The user terminal device 30 transmits the biological information measured by the biological information measurement device 31 to the information processing apparatus 10. Moreover, the user terminal device 30 transmits input information, which is input to the user terminal device 30, to the information processing apparatus 10. The input information may be information input on various applications installed in the user terminal device 30. For example, the input information may include the search keyword input to the search engine for searching for information on the network. Moreover, the input information may include a word transmitted or dispatched to a specified or unspecified party, such as a transmission mail, the message on the messenger application, dispatch on the SNS, description of the weblog, or a chat conversation.

The user terminal device 30 may immediately transmit these pieces of information to the information processing apparatus 10 after the biological information is measured and after the input information is input, or the user terminal device 30 may transmit these pieces of information to the information processing apparatus 10 at a predetermined time point. Moreover, the user terminal device 30 may transmit the biological information and the input information to the information processing apparatus 10 based on a transmission instruction of these pieces of information. The transmission instruction of the biological information and the input information may be transmitted from the information processing apparatus 10. The information processing apparatus 10 performs information processing for supporting the creation of the electronic medical record 51, by using the biological information and the input information transmitted from the user terminal device 30. Moreover, the user may be able to set any time for a transmission timing to the information processing apparatus 10.

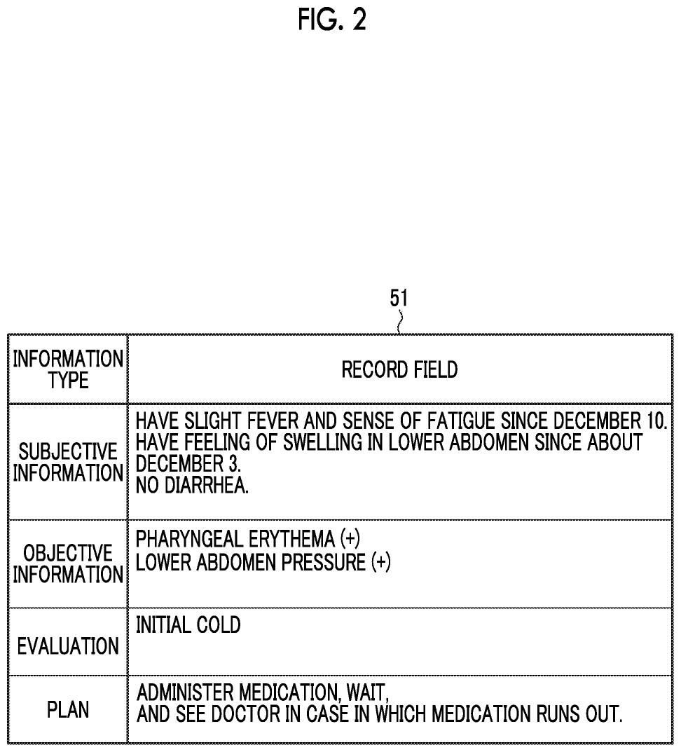

FIG. 2 is a diagram showing an example of the medical information recorded in the electronic medical record 51. The medical information recorded in the electronic medical record 51 includes “subjective information”, “objective information”, “evaluation”, and “plan”. The “subjective information” is information obtained through an interview with the patient, and examples thereof include information on a subjective symptom, an onset time, a current medical history, a past history, a family history, and a life history complained by the patient himself/herself.

The “objective information” is objective information obtained by performing the medical examination and examination on the patient. The “objective information” includes data indicating results of various examinations performed on the patient. The examination method includes various examination methods, such as an image examination, a sample examination, and a physiological function examination, and is typically formulated by a doctor in consideration of the subjective information and the like. An examination order is created based on the formulated examination method, and the examination is performed in accordance with the examination order. The examination order can be recorded as the medical information in the electronic medical record 51.

The “evaluation” is information indicating a comprehensive evaluation performed by an analysis or interpretation based on a diagnosis of a doctor, the subjective information, and the objective information. The “plan” is information indicating a treatment policy, a content, a lifestyle guide, and the like determined based on the “evaluation”. The electronic medical record 51 can be viewed, created, and edited by, for example, the doctor using the information processing apparatus 10. The created electronic medical record 51 is stored and saved in the medical data server 50.

As described above, the examination method for making the diagnosis for a body of the patient is typically formulated based on the subjective information or the like obtained through the interview with the patient. However, there is a case in which the patient is not clearly aware of his/her symptom or cannot correctly report his/her symptom to the doctor, and in this case, there is a risk that the information lacking in the accuracy is recorded in the electronic medical record 51 or the useful information is not recorded in the electronic medical record 51. In a case in which the medical information recorded in the electronic medical record 51 lacks in the accuracy or lacks in the information, it is difficult to formulate an appropriate examination method. The information processing apparatus 10 has a function of supporting the formulation of the examination method.

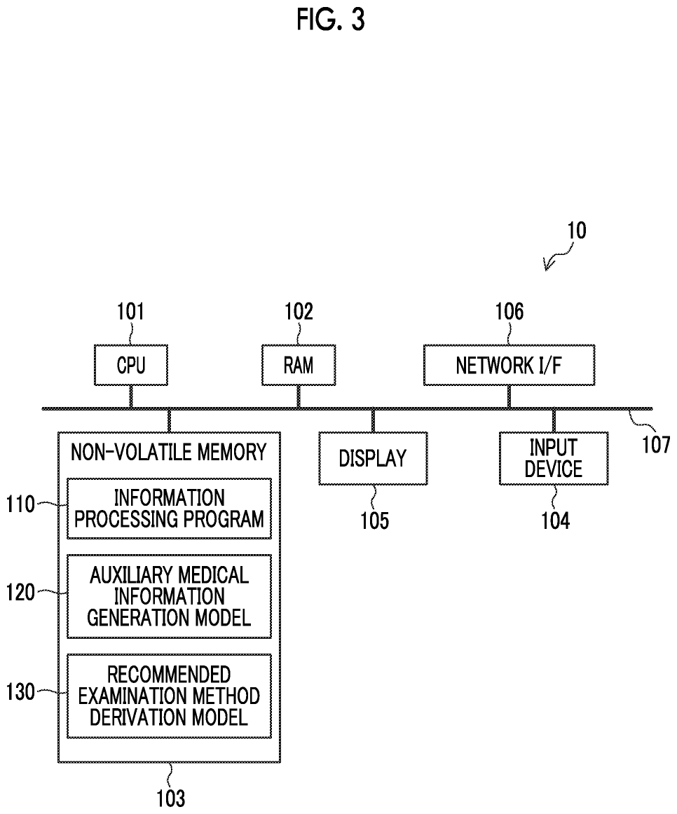

FIG. 3 is a diagram showing an example of a hardware configuration of the information processing apparatus 10. The information processing apparatus 10 includes a central processing unit (CPU) 101, a random access memory (RAM) 102, a non-volatile memory 103, an input device 104, a display 105, and a network interface 106. These pieces of hardware are connected to a bus 107.

For example, the display 105 is a liquid crystal display or a light emitting diode (LED) display. The input device 104 includes, for example, a keyboard and a mouse, and may include a near input device, such as a touch panel display, a voice input device, such as a microphone, and a contactless input device, such as a camera or a sensor. The network interface 106 is an interface for connecting the information processing apparatus 10 to the networks 41 and 42.

The non-volatile memory 103 is a non-volatile storage medium, such as a hard disk and a flash memory. An information processing program 110, an auxiliary medical information generation model 120, and a recommended examination method derivation model 130 are stored in the non-volatile memory 103. The RAM 102 is a work memory for the CPU 101 to execute processing. The CPU 101 loads the information processing program 110 stored in the non-volatile memory 103 into the RAM 102, and executes the processing in accordance with the information processing program 110. The CPU 101 is an example of a “processor” according to the disclosed technology. Details of the auxiliary medical information generation model 120 and the recommended examination method derivation model 130 will be described below.

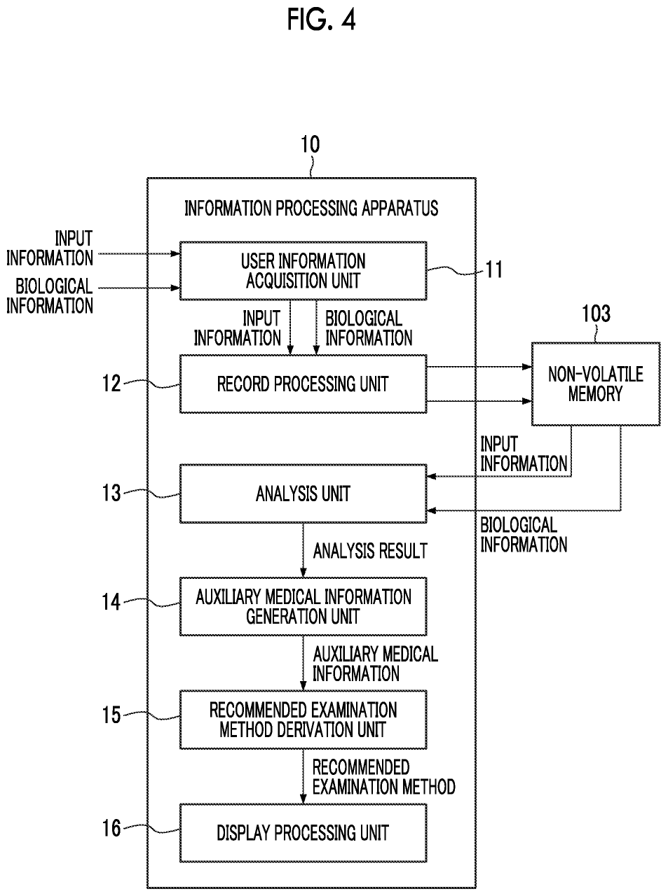

FIG. 4 is a functional block diagram showing an example of a functional configuration of the information processing apparatus 10. The information processing apparatus 10 includes a user information acquisition unit 11, a record processing unit 12, an analysis unit 13, an auxiliary medical information generation unit 14, a recommended examination method derivation unit 15, and a display processing unit 16. By the CPU 101 executing the information processing program 110, the CPU 101 functions as the user information acquisition unit 11, the record processing unit 12, the analysis unit 13, the auxiliary medical information generation unit 14, the recommended examination method derivation unit 15, and the display processing unit 16.

The user information acquisition unit 11 acquires the biological information and the input information of the user (patient) transmitted from the user terminal device 30. For example, the user information acquisition unit 11 acquires the biological information and the input information at a frequency of once or twice or more times a day.

The record processing unit 12 performs processing of giving metadata including date and time information indicating measurement date and time of the biological information and a user identification (ID) to the biological information acquired by the user information acquisition unit 11, and recording (saving) the biological information to which the metadata is given in the non-volatile memory 103. Moreover, the record processing unit 12 performs selection processing for selecting input information including a specific word related to a physical condition of the user (patient) out of the input information acquired by the user information acquisition unit 11. The record processing unit 12 performs processing of giving metadata including date and time information indicating input date and time of the input information and the user ID to the selected input information and recording (saving) the input information to which the metadata is given in the non-volatile memory 103.

Examples of the specific word related to the physical condition of the user (patient) include “fever”, “cough”, “headache”, “stomachache”, “dizziness”, “cold”, “sleeplessness”, “uneasiness”, “depressed”, “listless”, “annoyed”, “influenza”, “novel coronavirus”, and “COVID-19”. Such a specific word related to the physical condition of the user (patient) may be registered in advance. In this case, the record processing unit 12 may select the input information including the word registered in advance out of the input information acquired by the user information acquisition unit 11 and record the selected input information in the non-volatile memory 103. By performing the selection processing described above on the input information, the input information irrelevant to the physical condition of the user (patient) is excluded from a processing target. The biological information and the input information transmitted daily from the user terminal device 30 are accumulated in the non-volatile memory 103 of the information processing apparatus 10.

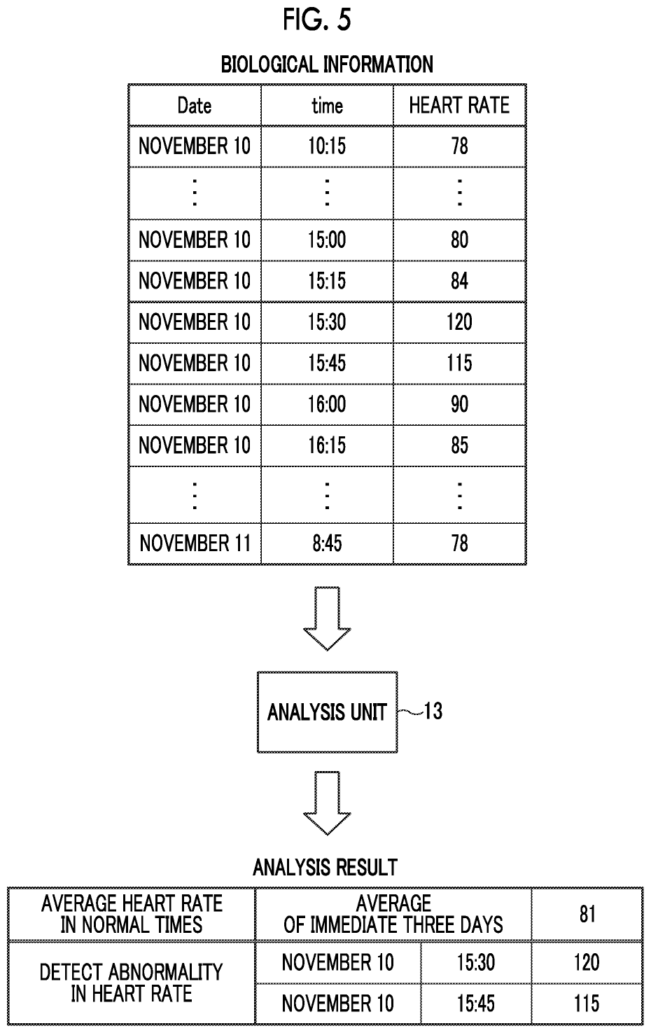

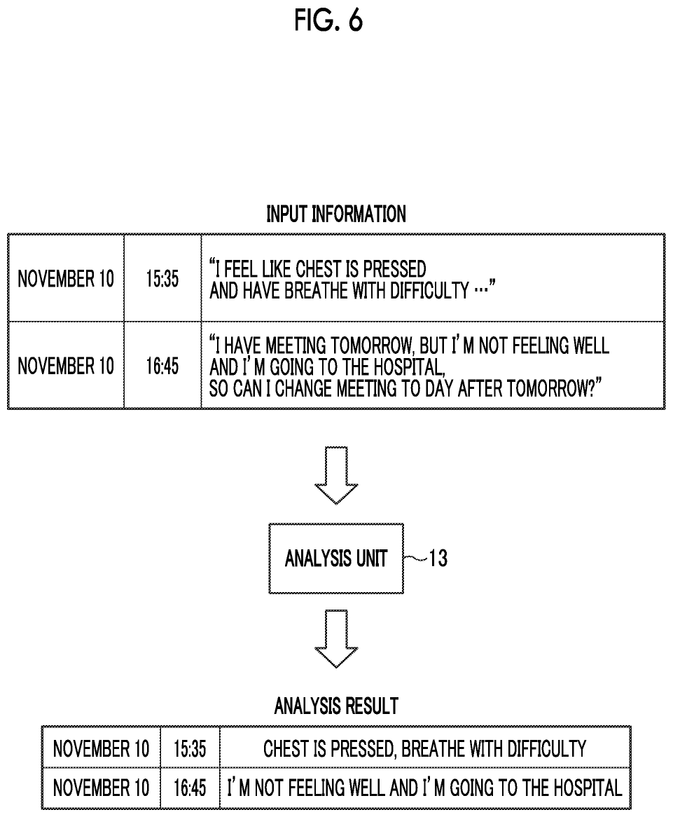

The analysis unit 13 performs analysis processing on the biological information and the input information recorded in the non-volatile memory 103 to extract information indicating that an unusual event has occurred in the physical condition of the user from the biological information and the input information. FIGS. 5 and 6 are diagrams showing an example of the analysis processing performed by the analysis unit 13, respectively. As shown in FIG. 5, the analysis unit 13 acquires the biological information recorded in the non-volatile memory 103. FIG. 5 shows time-series data of a heart rate as an example of the biological information. The analysis unit 13 calculates an average value of the heart rates in normal times in the last few days based on the time-series data of the heart rate. In a case in which the time-series data of the heart rate includes data in which a difference or a fluctuation rate with respect to the heart rate in normal times is equal to or larger than a predetermined value, the analysis unit 13 determines that the heart rate is abnormal, and extracts the data indicating the abnormal value. FIG. 5 shows a case in which each measurement value (120, 115) at 15:30 and 15:45 on November 10 is extracted as the data indicating the abnormality.

Moreover, as shown in FIG. 6, the analysis unit 13 acquires the input information recorded in the non-volatile memory 103. FIG. 6 shows a message which is transmitted from the user and input to the messenger application, as an example of the input information. The analysis unit 13 extracts a word indicating the unusual event in the physical condition of the user, which is included in the input information. FIG. 6 shows a case in which words of “chest is pressed” and “breathe with difficulty” are extracted from the message input at 15:35 on November 10 and a word of “I'm not feeling well and I'm going to the hospital” is extracted from the message input at 16:45 on the same day. The analysis unit 13 derives the information, which is extracted from the biological information and the input information, as an analysis result.

The auxiliary medical information generation unit 14 generates the auxiliary medical information based on the analysis result derived by the analysis unit 13. The auxiliary medical information is auxiliary information for the medical information (subjective information and objective information) recorded in the electronic medical record 51, and is information indicating the physical condition of the user based on the biological information and the input information. The auxiliary medical information generation unit 14 generates the auxiliary medical information described in the same form as a recording form of the medical information recorded in the electronic medical record 51.

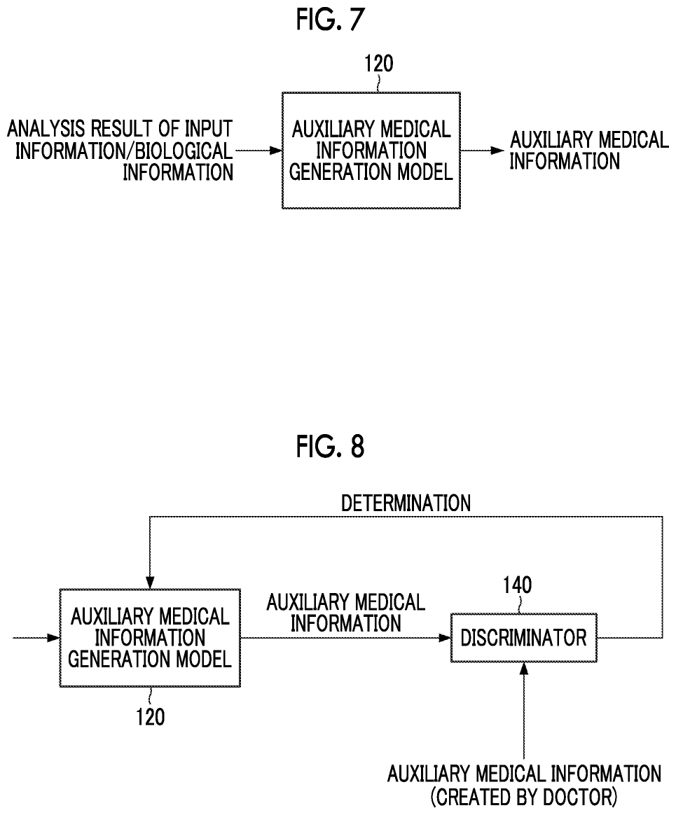

As shown in FIG. 7, the auxiliary medical information generation unit 14 generates the auxiliary medical information by using the auxiliary medical information generation model 120 stored in the non-volatile memory 103. The auxiliary medical information generation model 120 is a mathematical model in which the analysis result of the input information and the biological information is used as input and the auxiliary medical information is used as output, and is constructed by machine learning.

FIG. 8 is a diagram showing an example of a training method of the auxiliary medical information generation model 120. The auxiliary medical information generation model 120 is trained using a so-called generative adversarial networks (GAN), for example. That is, a discriminator 140 is used for training the auxiliary medical information generation model 120. The discriminator 140 determines whether the auxiliary medical information input to the discriminator 140 is generated by the auxiliary medical information generation model 120 (fake) or created by the doctor (real). The auxiliary medical information generation model 120 is trained by error back propagation based on a determination result by the discriminator 140. The auxiliary medical information generation model 120 is trained until the auxiliary medical information (fake) generated by the auxiliary medical information generation model 120 is erroneously determined by the discriminator 140 as being created by the doctor (real). On the other hand, the discriminator 140 is trained to be able to correctly determine whether the auxiliary medical information input to the discriminator 140 is generated by the auxiliary medical information generation model 120 (fake) or created by the doctor (real). It should be noted that the auxiliary medical information generation model 120 may be constructed by the machine learning using a plurality of combinations of the analysis result of the input information and the biological information for training and the auxiliary medical information for training as training data.

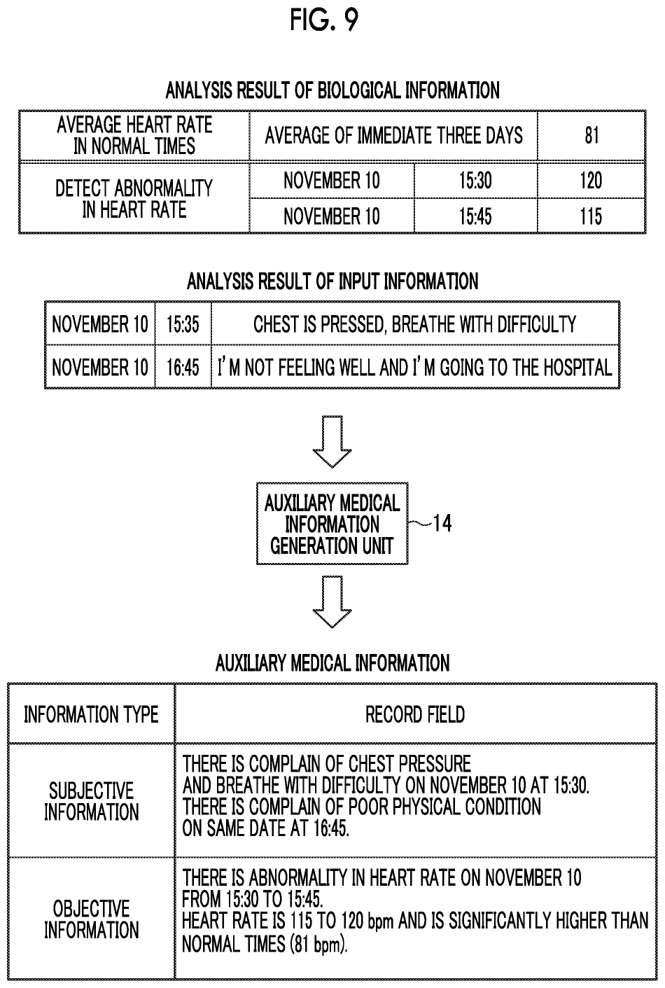

FIG. 9 is a diagram showing an example of generation processing performed by the auxiliary medical information generation unit 14. FIG. 9 shows a case in which the auxiliary medical information is generated based on the analysis result by the analysis unit 13 shown in FIGS. 5 and 6. As shown in FIG. 9, the auxiliary medical information may include auxiliary subjective information generated based on the input information and auxiliary objective information generated based on the biological information.

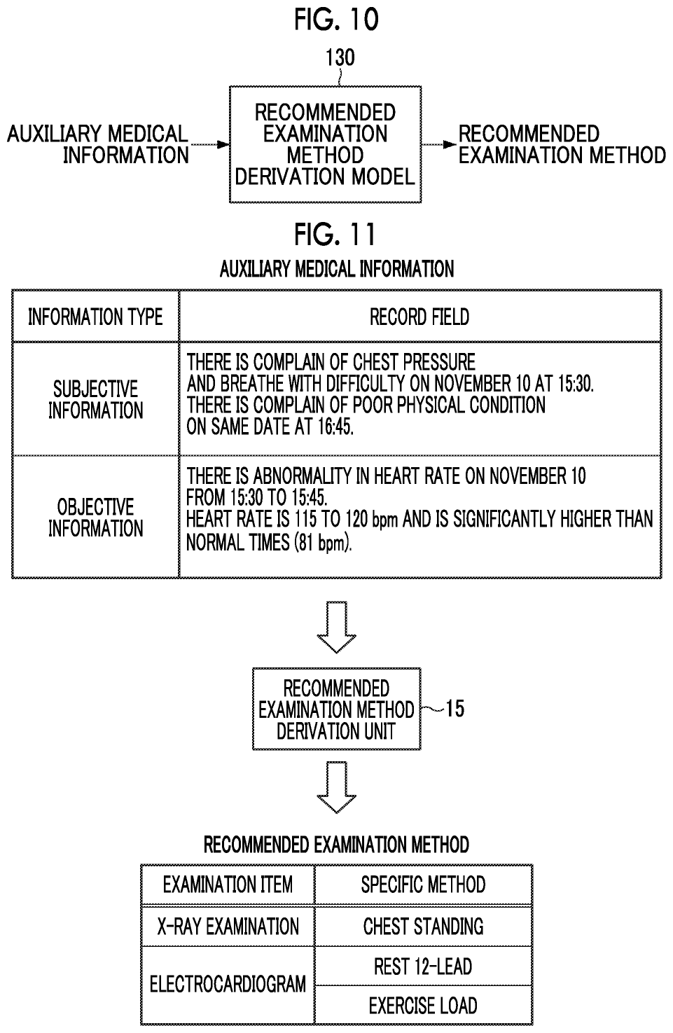

The recommended examination method derivation unit 15 derives the examination method for making the diagnosis on the body of the user as the recommended examination method based on the auxiliary medical information generated by the auxiliary medical information generation unit 14. As shown in FIG. 10, the recommended examination method derivation unit 15 derives the recommended examination method by using the recommended examination method derivation model 130 stored in the non-volatile memory 103. The recommended examination method derivation unit 15 is a mathematical model, such as a neural network in which the auxiliary medical information is used as input and the recommended examination method is used as output. The recommended examination method derivation model 130 is constructed, for example, by the machine learning using a plurality of combinations of the auxiliary medical information for training and the recommended examination method for training as the training data. As the recommended examination method for training, for example, a method selected as the examination method deemed necessary for an experienced and skilled doctor to estimate a cause, a disease name, an abnormal part, and the like from the symptom indicated by the corresponding auxiliary medical information for training and to make a definitive diagnosis of these can be used.

FIG. 11 is a diagram showing an example of derivation processing performed by the recommended examination method derivation unit 15. FIG. 11 shows a case in which the recommended examination method is derived based on the auxiliary medical information shown in FIG. 9. As shown in FIG. 9, the recommended examination method may include examination items (for example, X-ray examination, electrocardiogram) and specific methods (for example, chest standing, rest 12-lead, exercise load).

The display processing unit 16 displays the recommended examination method, which is derived by the recommended examination method derivation unit 15, on the display 105. For example, in a case in which the display processing unit 16 creates the examination order by using the information processing apparatus 10, the display processing unit 16 may display the recommended examination method for the patient. Moreover, the display processing unit 16 may display the recommended examination method on the display 105 together with the corresponding auxiliary medical information.

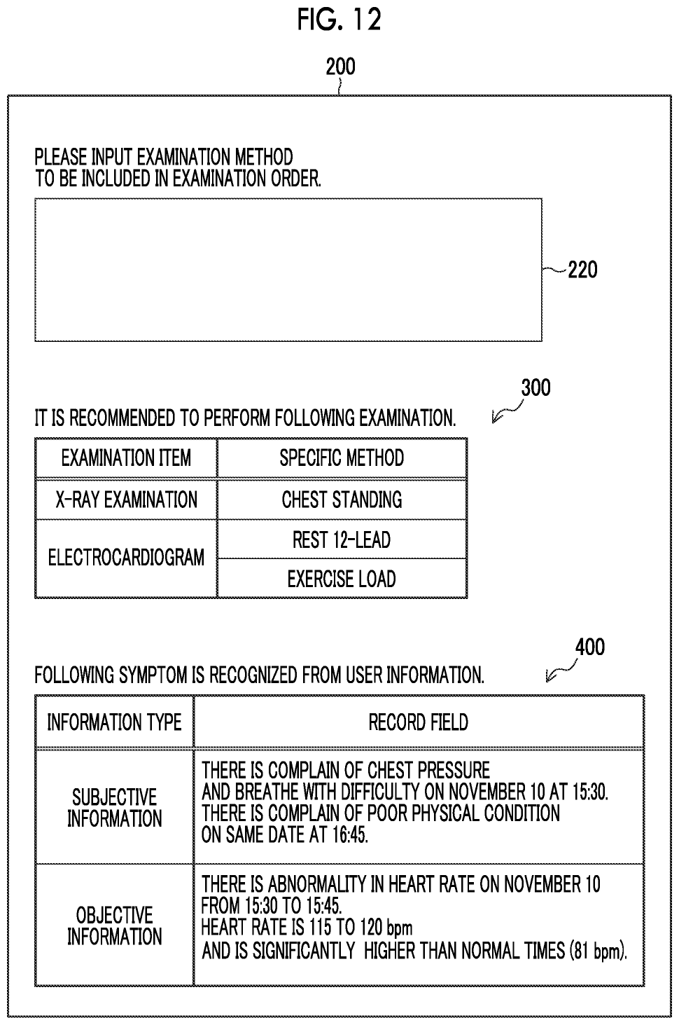

FIG. 12 is a diagram showing an example of a display form of the recommended examination method. As shown in FIG. 12, in a case in which the examination order is created, the display processing unit 16 displays an input field 220 of the examination method included in the examination order on the display screen 200 of the display 105. Moreover, the display processing unit 16 displays a recommended examination method 300 derived by the recommended examination method derivation unit 15 and auxiliary medical information 400 generated by the auxiliary medical information generation unit 14 on the display screen 200 together with the input field 220 of the examination method. The doctor can input the examination method to be included in the examination order in the input field 220 with reference to the recommended examination method 300 and the auxiliary medical information 400 displayed on the display screen 200 as well as the medical information recorded in the electronic medical record 51. It should be noted that the input method of the examination method may be a method of directly inputting a text or a method of selecting one or more examination methods from among a plurality of examination methods included in a pull-down menu.

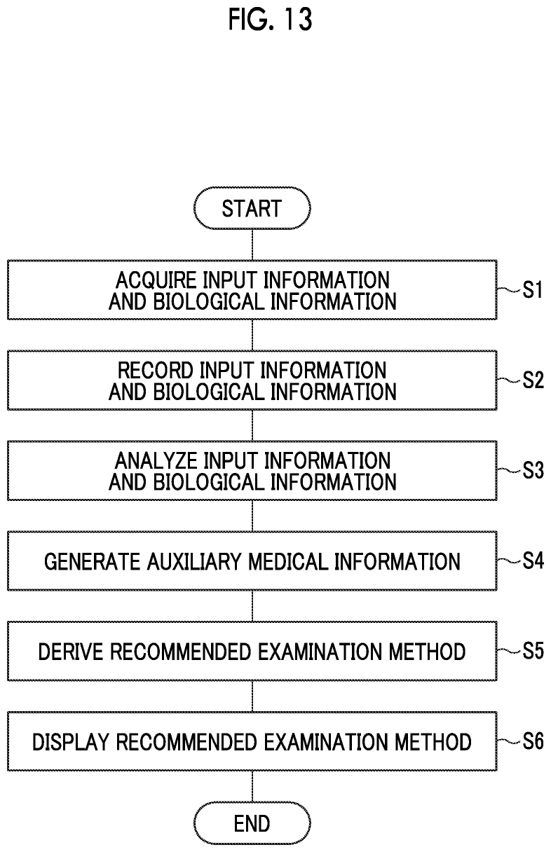

FIG. 13 is a flowchart showing an example of a flow of processing performed by the CPU 101 executing the information processing program 110. The information processing program 110 is executed, for example, in a case in which the user instructs the start of processing by operating the input device 104.

In step S1, the CPU 101 functions as the user information acquisition unit 11 to acquire the biological information and the input information of the user (patient) which are transmitted from the user terminal device 30. The CPU 101 acquires the biological information and the input information at, for example, a frequency of once or twice or more times a day.

In step S2, the CPU 101 functions as the record processing unit 12 to perform processing of giving the metadata including the date and time information indicating the measurement date and time and the user ID in the biological information acquired in step S1 and recording (saving) the biological information to which the metadata is given in the non-volatile memory 103. Moreover, the CPU 101 performs the selection processing of selecting the input information including the specific word related to the physical condition of the user (patient) out of the input information acquired in step S1 performs the processing of giving the metadata including the date and time information indicating the input date and time of the input information and the user ID to the selected input information and recording (saving) the input information to which the metadata is given in the non-volatile memory 103. The biological information and the input information transmitted daily from the user terminal device 30 are accumulated in the non-volatile memory 103 of the information processing apparatus 10.

In step S3, the CPU 101 functions as the analysis unit 13 to perform the analysis processing on the biological information and the input information recorded in the non-volatile memory 103 to extract the information indicating that the unusual event has occurred in the physical condition of the user from the biological information and the input information. The analysis unit 13 derives the information, which is extracted from the biological information and the input information, as the analysis result.

In step S4, the CPU 101 functions as the auxiliary medical information generation unit 14 to generate the auxiliary medical information based on the analysis result derived in step S3. The CPU 101 generates the auxiliary medical information by inputting the analysis result of the input information and the biological information to the auxiliary medical information generation model 120 stored in the non-volatile memory 103. The auxiliary medical information may include the auxiliary subjective information generated based on the input information and the auxiliary objective information generated based on the biological information.

In step S5, the CPU 101 functions as the recommended examination method derivation unit 15 to derive the examination method for making the diagnosis for the body of the user as the recommended examination method based on the auxiliary medical information generated in step S4. The CPU 101 derives the recommended examination method by inputting the auxiliary medical information to the recommended examination method derivation model 130 stored in the non-volatile memory 103.

In step S6, the CPU 101 functions as the display processing unit 16 to display the recommended examination method derived in step S5 on the display screen 200 of the display 105. The CPU 101 may display the recommended examination method for the patient, for example, in a case in which the examination order is recorded in the electronic medical record 51 by using the information processing apparatus 10. Moreover, the display processing unit 16 may display the recommended examination method on the display 105 together with the corresponding auxiliary medical information generated in step S4.

As described above, the information processing apparatus 10 acquires the input information, which is input to the user terminal device used by the user (patient), and the biological information measured for the user (patient), and derives the examination method for making the diagnosis for the body of the user as the recommended examination method based on the biological information and the input information. The information processing apparatus 10 presents, for example, the recommended examination method in a case in which the examination order is created for the user (patient).

As described above, there is a case in which the information lacking in the accuracy is recorded in the electronic medical record 51 or the useful information is not recorded in the electronic medical record 51, and in such a case, it is difficult to formulate an appropriate examination method. With the information processing apparatus 10 according to the embodiment of the disclosed technology, the recommended examination method is derived based on the biological information and the input information. The biological information and the input information are information acquired on a daily basis for the user, and may include the auxiliary information for the medical information recorded in the electronic medical record 51. It can be said that the recommended examination method derived based on such biological information and input information has a certain degree of validity. That is, with the information processing apparatus 10 according to the embodiment of the disclosed technology, it is possible to support the formulation of the examination method for making the diagnosis for the body of the user.

It should be noted that, in the present embodiment, the case has been described in which the recommended examination method is derived based on both the biological information and the input information is shown, but at least one of the biological information or the input information may be acquired to derive the recommended examination method based on the acquired biological information or input information.

Moreover, in the present embodiment, the case has been described in which the information processing apparatus 10 acquires the biological information and the input information transmitted from the user terminal device 30, but in a case in which the biological information and the input information are accumulated in a predetermined server, the information processing apparatus 10 may access the server and acquire the biological information and the input information.

Moreover, the case has been described in which the information processing apparatus 10 has the functions of the record processing unit 12, the analysis unit 13, the auxiliary medical information generation unit 14, and the recommended examination method derivation unit 15, but the user terminal device 30 may have at least a part of these functions.

### Second Embodiment

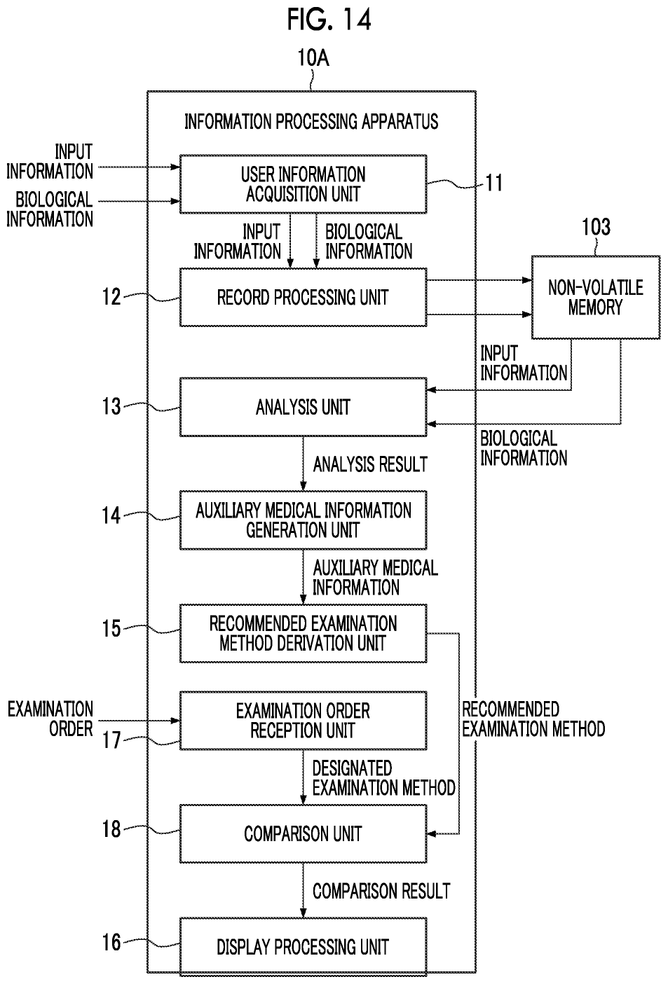

FIG. 14 is a functional block diagram showing an example of a functional configuration of an information processing apparatus 10A according to a second embodiment of the disclosed technology. The information processing apparatus 10A according to the present embodiment is different from the information processing apparatus 10 according to the first embodiment (see FIG. 4) in that an examination order reception unit 17 and a comparison unit 18 are further provided.

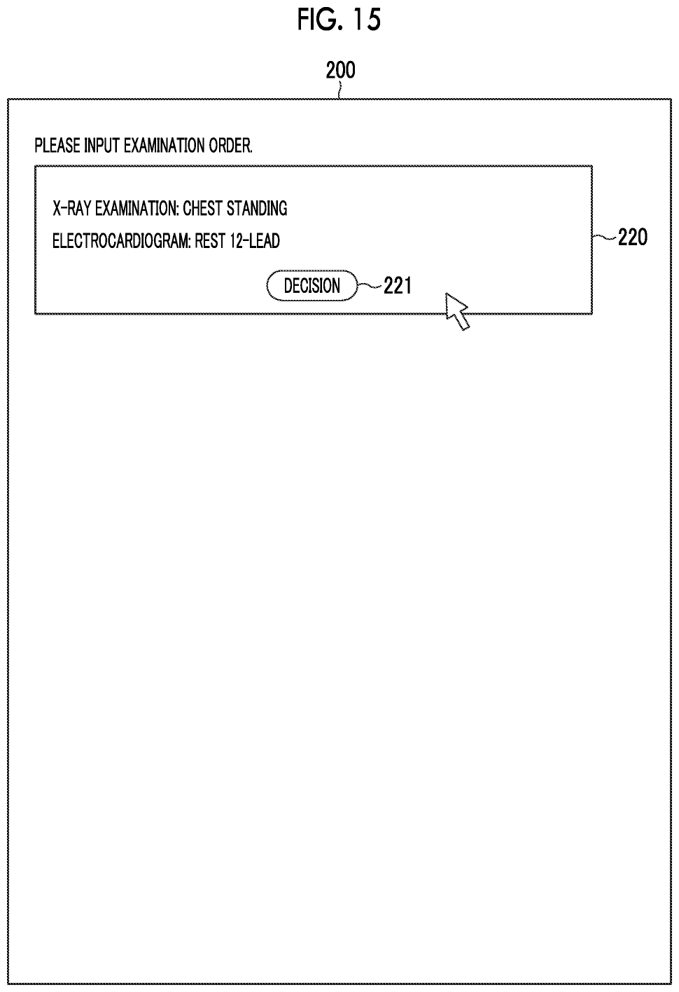

The examination order reception unit 17 receives designation of the examination method to be included in the examination order for the user (patient). The examination order reception unit 17 displays an examination order reception screen shown in FIG. 15 on the display screen 200 of the display 105. The doctor who creates the examination order inputs the examination method to be included in the examination order in the input field 220 in consideration of the subjective information and the like recorded in the electronic medical record 51. The input method of the examination method may be the method of directly inputting the text or the method of selecting one or more examination methods from among the plurality of examination methods included in the pull-down menu. In a case in which the examination method is input to the input field 220 and a decision button 221 is clicked, the examination method input to the input field 220 is designated as the examination method to be included in the examination order (hereinafter, referred to as a designated examination method), and is received by the examination order reception unit 17.

The comparison unit 18 compares the designated examination method received by the examination order reception unit 17 with the recommended examination method derived by the recommended examination method derivation unit 15 to derive the comparison result. Specifically, the comparison unit 18 discriminates, among the recommended examination methods, a recommended examination method that does not match the designated examination method and a recommended examination method that matches the designated examination method.

The display processing unit 16 displays the recommended examination method derived by the recommended examination method derivation unit 15 on the display screen 200 of the display 105 together with the comparison result derived by the comparison unit 18. More specifically, among the recommended examination methods, the recommended examination method that matches the designated examination method and the recommended examination method that does not match the designated examination method are displayed on the display screen 200 in a distinguishable manner.

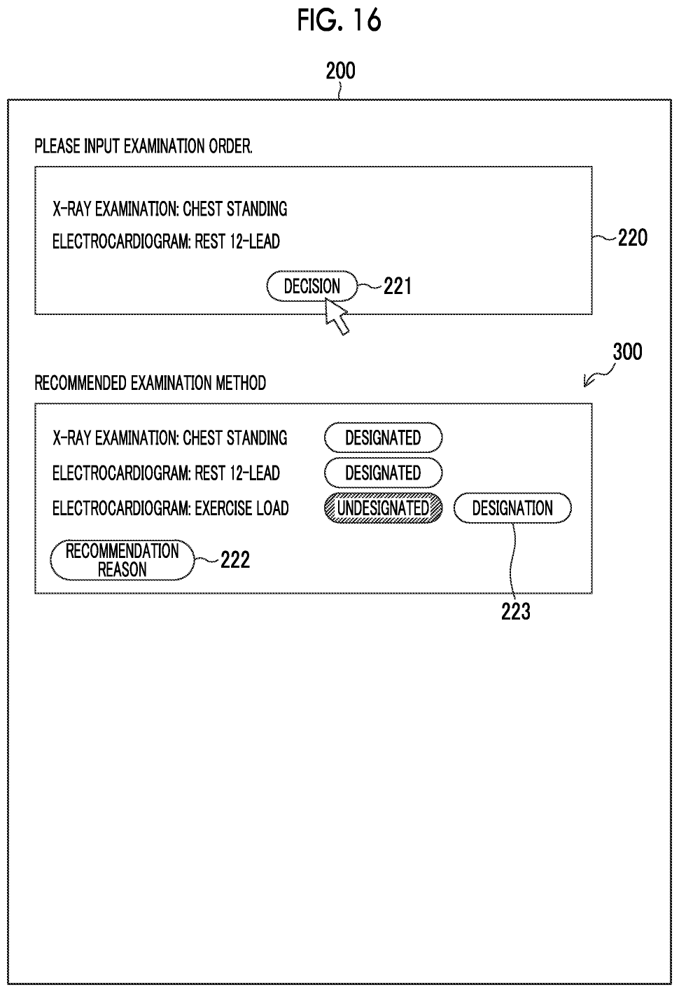

FIG. 16 is a diagram showing an example of a display aspect of the comparison result by the comparison unit 18. In a case in which the examination method is input to the examination method input field 220 and the decision button 221 is clicked, the display processing unit 16 displays the recommended examination method 300 derived by the recommended examination method derivation unit 15 on the display screen. Among the recommended examination methods, the display processing unit 16 displays “designated” for the recommended examination method that matches the designated examination method, and displays “undesignated” for the recommended examination method that does not match the designated examination method. In the example shown in FIG. 16, “X-ray examination: chest standing” and “electrocardiogram: rest 12-lead” derived as the recommended examination methods match the designated examination method, and “designated” is displayed for these examination methods. On the other hand, the “electrocardiogram: exercise load” derived as the recommended examination method does not match the designated examination method, and “undesignated” is displayed for this examination method.

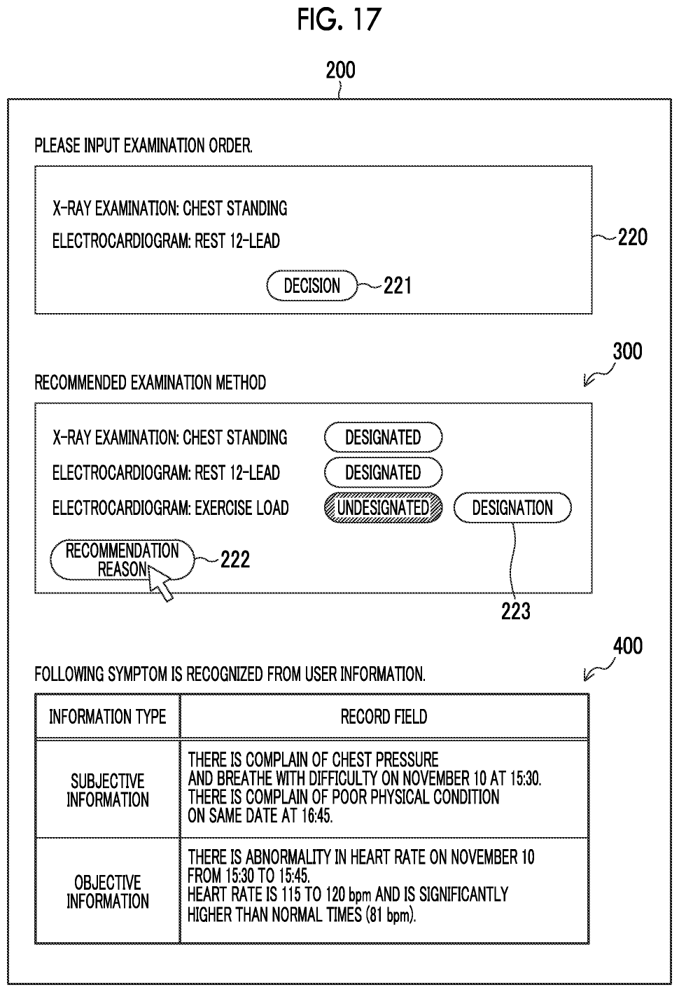

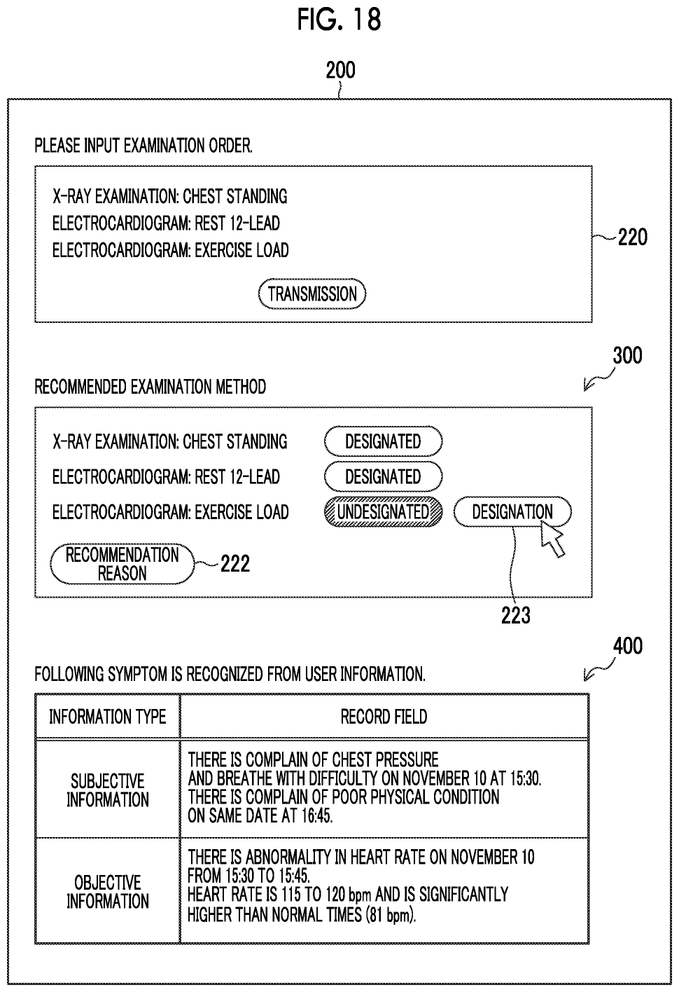

As shown in FIG. 17, in a case in which a recommendation reason button 222 is clicked, the display processing unit 16 displays the auxiliary medical information 400 generated by the auxiliary medical information generation unit 14 on the display screen 200. The doctor can determine the validity of the recommended examination method 300 with reference to the auxiliary medical information 400. Moreover, as shown in FIG. 18, by clicking the designation button 223 for the recommended examination method of “undesignated”, which is not designated as the examination method including the examination order, it is possible to designate the examination method as the examination method including the examination order.

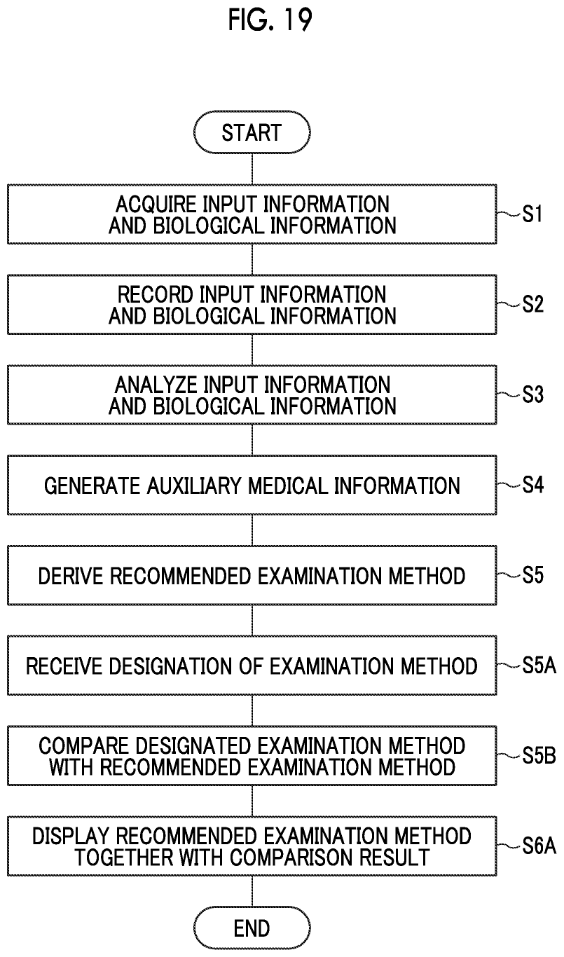

FIG. 19 is a flowchart showing an example of a flow of processing performed by the CPU 101 according to the present embodiment executing the information processing program 110. In the present flowchart, steps S5A and S5B are added to the flowchart shown in FIG. 13, and step S6 is changed to S6A. In the following, only steps different from the flowchart shown in FIG. 13 will be described, and the description of the other steps will be omitted.

In step S5A, the CPU 101 functions as the examination order reception unit 17 to receive the designation of the examination method to be included in the examination order for the user (patient). In step S5B, the CPU 101 functions as the comparison unit 18 to compare the designated examination method received in step S5A with the recommended examination method derived in step S5 and derive the comparison result. Specifically, the CPU 101 discriminates, among the recommended examination methods, the recommended examination method that does not match the designated examination method and the recommended examination method that matches the designated examination method. It should be noted that the processing in this step S5A may be performed in parallel with any processing of steps S1 to S5, or may be performed before the processing of steps S1 to S5.

In step S6A, the CPU 101 functions as the display processing unit 16 to display the recommended examination method derived in step S5 on the display screen 200 of the display 105 together with the comparison result derived in step S5A. More specifically, among the recommended examination methods, the recommended examination method that matches the designated examination method and the recommended examination method that does not match the designated examination method are displayed on the display screen 200 in a distinguishable manner.

As described above, with the information processing apparatus 10A according to the present embodiment, among the recommended examination methods, the recommended examination method that matches the designated examination method and the recommended examination method that does not match the designated examination method are presented in a distinguishable manner. That is, since it is possible to clearly indicate which of the recommended examination methods is not included in the examination order, it is possible to improve the effect of supporting the formulation of the examination method.

### Third Embodiment

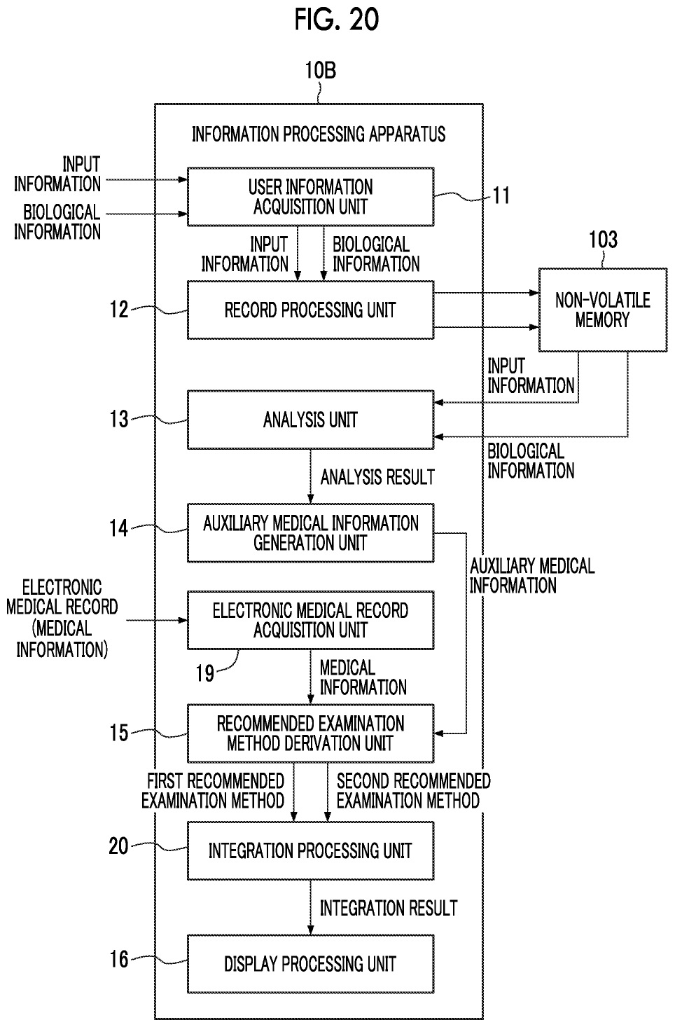

FIG. 20 is a functional block diagram showing an example of a functional configuration of an information processing apparatus 10B according to a third embodiment of the disclosed technology. The information processing apparatus 10B according to the present embodiment is different from the information processing apparatus 10 according to the first embodiment (see FIG. 4) in that an electronic medical record acquisition unit 19 and an integration processing unit 20 are further provided.

The electronic medical record acquisition unit 19 acquires the electronic medical record 51 in which the medical information is recorded. For example, the electronic medical record acquisition unit 19 may acquire the electronic medical record 51 in a case in which the creation of the electronic medical record 51 using the information processing apparatus 10 is completed. Moreover, the electronic medical record acquisition unit 19 may acquire the created electronic medical record 51 stored in the medical data server 50.

In the present embodiment, the recommended examination method derivation unit 15 derives a first recommended examination method based on the auxiliary medical information generated by the auxiliary medical information generation unit 14. Moreover, the recommended examination method derivation unit 15 derives a second recommended examination method based on the medical information recorded in the electronic medical record 51 acquired by the electronic medical record acquisition unit 19.

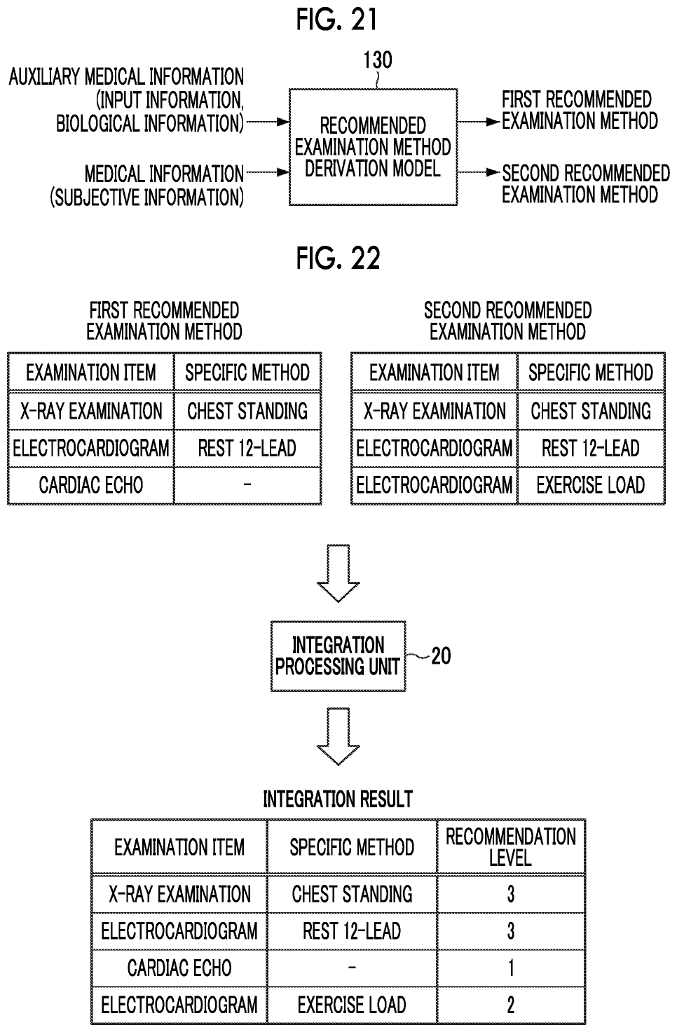

As shown in FIG. 21, the recommended examination method derivation unit 15 derives the first and second recommended examination methods by using the recommended examination method derivation model 130 stored in the non-volatile memory 103. In the present embodiment, the recommended examination method derivation model 130 derives the first recommended examination method based on the auxiliary medical information including the biological information and the input information, and derives the second recommended examination method based on the medical information recorded in the electronic medical record 51. The medical information recorded in the electronic medical record 51 includes the subjective information obtained through the interview with the medical information patient. That is, the recommended examination method derivation model 130 derives the second recommended examination method in consideration of the information on the subjective symptom, the onset time, the current medical history, the past history, the family history, and the life history complained by the patient himself/herself.

The integration processing unit 20 performs integration processing of integrating the examination method derived as the first recommended examination method and the examination method derived as the second recommended examination method. For each of the examination method derived as the first recommended examination method and the examination method derived as the second recommended examination method, the integration processing unit 20 gives information indicating a degree of recommendation depending on whether the examination method is included in the first or second recommended examination method. Specifically, the integration processing unit 20 gives the information indicating that the degree of recommendation is high to the examination method included in both the first recommended examination method and the second recommended examination method. Moreover, the integration processing unit 20 gives the information indicating that the degree of recommendation is moderate to the examination method that is not included in the first recommended examination method and is included in the second recommended examination method. Moreover, the integration processing unit 20 gives the information indicating that the degree of recommendation is low to the examination method that is included in the first recommended examination method and is not included in the second recommended examination method. The integration processing unit 20 derives, as an integration result, the recommended examination method to which information indicating the degree of recommendation is given.

FIG. 22 is a diagram showing an example of the integration processing in the integration processing unit 20. FIG. 22 shows an example in which a recommendation level 3 is given for “X-ray examination: chest standing” and “electrocardiogram: rest 12-lead” included in both the first recommended examination method and the second recommended examination method, a recommendation level 1 is given to “cardiac echo” included only in the first recommended examination method, and a recommendation level 2 is given to “electrocardiogram: exercise load” included only in the second recommended examination method. In this example, a numerical value (recommendation level) is used as the information indicating the degree of recommendation, the recommendation level 3 indicates that the degree of recommendation is high, the recommendation level 2 indicates that the degree of recommendation is moderate, and the recommendation level 1 indicates that the degree of recommendation is low.

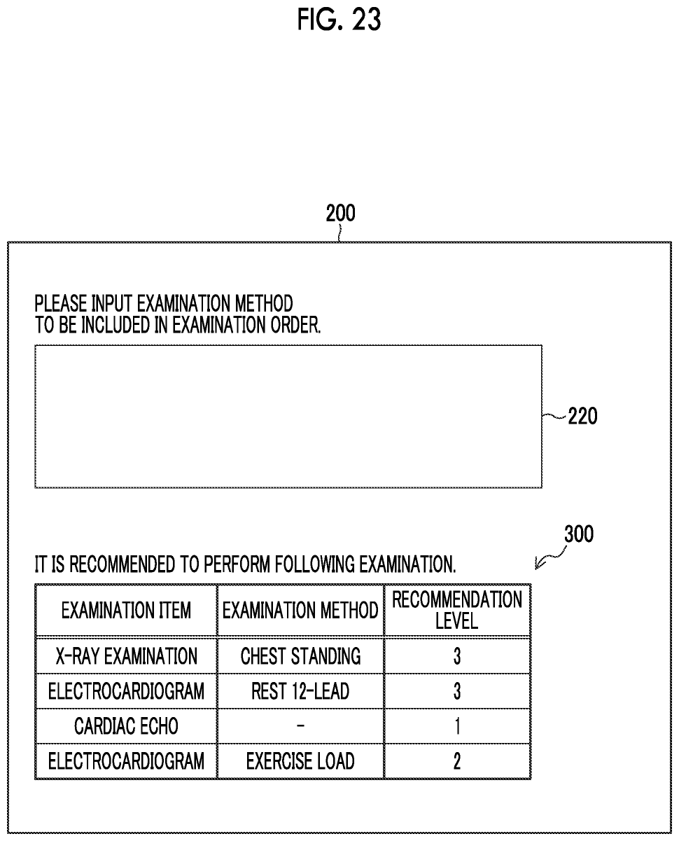

The display processing unit 16 displays the recommended examination method subjected to the integration processing by the integration processing unit 20 on the display 105. FIG. 23 is a diagram showing an example of a display form of the recommended examination method subjected to the integration processing. FIG. 23 shows the recommended examination method 300 in which the first recommended examination method or the second recommended examination method are integrated. The recommendation level is given as the information indicating the degree of recommendation to each examination method derived as each of the first recommended examination method or the second recommended examination method.

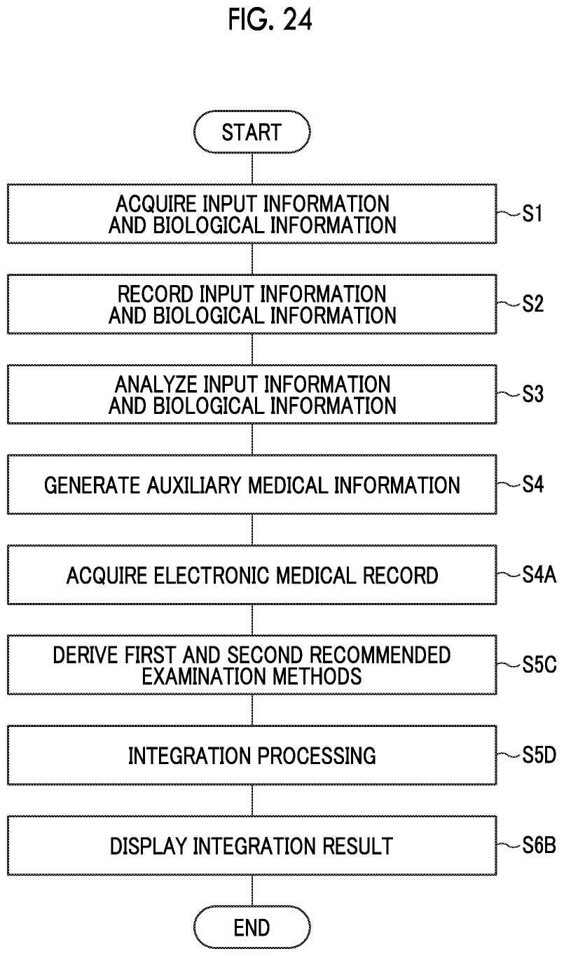

FIG. 24 is a flowchart showing an example of a flow of processing performed by the CPU 101 according to the present embodiment executing the information processing program 110. In the present flowchart, steps S4A and S5D are added to the flowchart shown in FIG. 13, step S5 is changed to step 5C, and step S6 is changed to S6B. In the following, only steps different from the flowchart shown in FIG. 13 will be described, and the description of the other steps will be omitted.

In step S4A, the CPU 101 functions as the electronic medical record acquisition unit 19 to acquire the electronic medical record 51 in which the medical information is recorded.

In step S5C, the CPU 101 functions as the recommended examination method derivation unit 15 to derive the first recommended examination method based on the auxiliary medical information generated in step S4, and derive the second recommended examination method based on the medical information recorded in the electronic medical record 51 acquired in step S4A. It should be noted that the processing in step S4A may be performed in parallel with any processing of steps S1 to S4, or may be performed before the processing of steps S1 to S4. Moreover, the derivation of the second recommended examination method in step S5C may be performed in parallel with the processing of steps S1 to S4 after the processing of step S4A.

In step S5D, the CPU 101 functions as the integration processing unit 20 to perform the integration processing of integrating the examination method derived as the first recommended examination method and the examination method derived as the second recommended examination method, and give the information indicating the degree of recommendation to each integrated examination method. Specifically, the CPU 101 gives the information indicating that the degree of recommendation is high to the examination method included in both the first recommended examination method and the second recommended examination method. Moreover, the CPU 101 gives the information indicating that the degree of recommendation is moderate to the examination method that is not included in the first recommended examination method and is included in the second recommended examination method. Moreover, the CPU 101 gives the information indicating that the degree of recommendation is low to the examination method that is included in the first recommended examination method and is not included in the second recommended examination method. The integration processing unit 20 derives, as an integration result, the recommended examination method to which information indicating the degree of recommendation is given.

In step S6B, the CPU 101 functions as the display processing unit 16 to display the integration result derived in step S5D on the display screen 200 of the display 105. The CPU 101 may display the first recommended examination method and the second recommended examination method, which are subjected to the integration processing, for the patient, for example, in a case in which the examination order is recorded in the electronic medical record 51 by using the information processing apparatus 10.

As described above, with the information processing apparatus 10B according to the present embodiment, the recommended examination method is derived based on the medical information recorded in the electronic medical record 51 as well as the biological information and the input information. The medical information recorded in the electronic medical record 51 includes the subjective information, such as the subjective symptom, the onset time, the current medical history, the past history, the family history, and the life history complained by the patient himself/herself. By including these pieces of the medical information recorded in the electronic medical record 51 in the information used for deriving the recommended examination method, a more appropriate recommended examination method can be derived.

Moreover, since the information indicating the degree of recommendation is given to each of the examination methods derived as the recommended examination method, it can be used as a reference in a case in which the examination method to be included in the examination order is designated from among the recommended examination method.

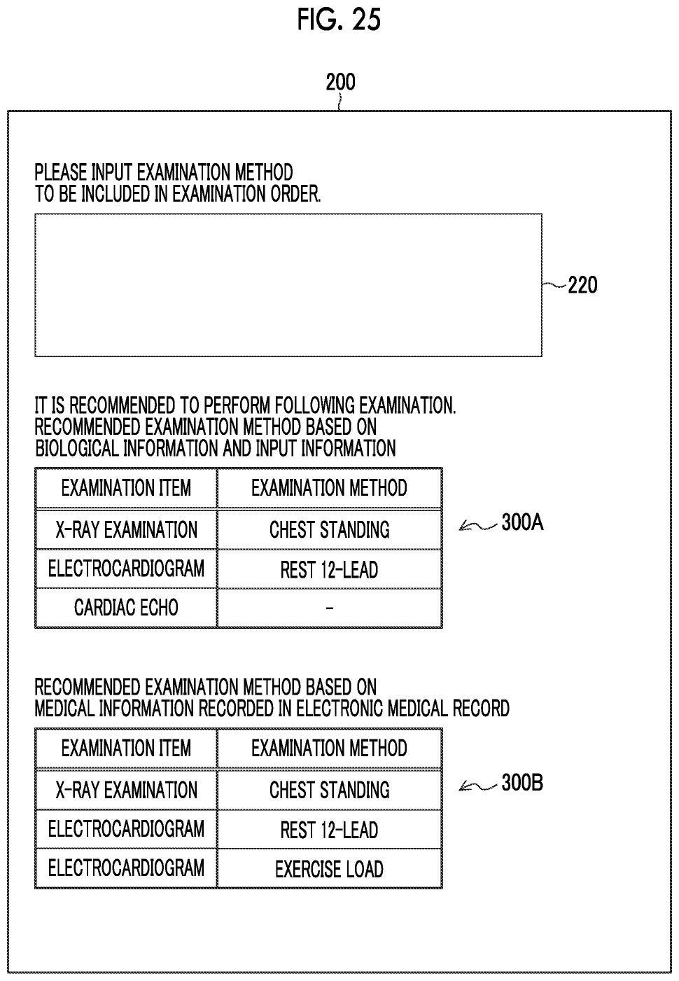

It should be noted that, in the present embodiment, the form has been described in which the result of integrating the first recommended examination method and the second recommended examination method is displayed on the display screen 200, but the disclosed technology is not limited to this aspect. As shown in FIG. 25, a first recommended examination method 300A based on the biological information and the input information and a second recommended examination method 300B based on the medical information recorded in the electronic medical record 51 may be displayed side by side on the display screen 200. In this case, the information processing apparatus 10B does not need to have a function of the integration processing unit 20.

The information processing apparatuses 10, 10A, and 10B according to the first to third embodiments described above are examples of an “information processing apparatus” according to the technology of the present disclosure. Moreover, the “information processing apparatus” according to the technology of the present disclosure may be a system including the configurations of the information processing apparatus 10, 10A, or 10B and the medical data server 50. More specifically, a system including the medical data server 50 and the information processing apparatus 10, 10A, or 10B which include at least the electronic medical record 51 may be used as the information processing apparatus. Moreover, in the present embodiment, the acquisition of the user information is periodically performed at a frequency of once or twice or more times a day, but the disclosed technology is not limited to this. For example, the user information acquisition unit 11 may start the acquisition of the user information by using a timing of accessing the user information in the electronic medical record or starting the input of the electronic medical record by the user as a trigger in a case in which the user visits the hospital.

Moreover, in the first to third embodiments described above, the electronic medical record has been described as the medical document data in which the medical information is recorded, but the recommended examination method may be derived based on the medical information recorded in the medical document data other than the electronic medical record.

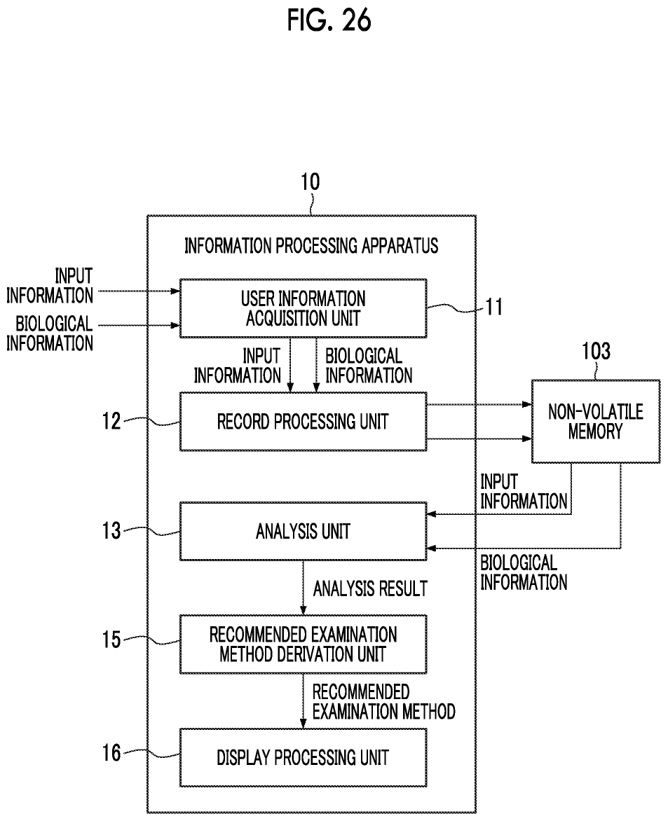

Moreover, in the first to third embodiments described above, the case has been described in which the recommended examination method derivation unit 15 derives the recommended examination method based on the auxiliary medical information generated by the auxiliary medical information generation unit 14, but the disclosed technology is not limited to this aspect. The recommended examination method derivation unit 15 may derive the recommended examination method based on the analysis result derived by the analysis unit 13. FIG. 26 is a functional block diagram showing an example of the functional configuration of the information processing apparatus 10 in this case. The recommended examination method derivation unit 15 may derive the recommended examination method, for example, by using a mathematical model, such as a neural network in which the analysis result (that is, information extracted from the biological information and the input information) by the analysis unit 13 is used as input and the recommended examination method is used as output.

In the embodiments described above, for example, as a hardware structure of processing units that execute various pieces of processing, such as the user information acquisition unit 11, the record processing unit 12, the analysis unit 13, the auxiliary medical information generation unit 14, the recommended examination method derivation unit 15, the display processing unit 16, the examination order reception unit 17, the comparison unit 18, the electronic medical record acquisition unit 19, and the integration processing unit 20, various processors described below can be used. The various processors include, as described above, in addition to the CPU and the GPU, which are general-purpose processors which execute software (program) and function as various processing units, a programmable logic device (PLD) which is a processor whose circuit configuration can be changed after manufacture, such as an FPGA, and a dedicated electric circuit which is a processor having a circuit configuration which is designed for exclusive use in order to execute specific processing, such as an application specific integrated circuit (ASIC).

One processing unit may be composed of one of the various processors or may be composed of a combination of two or more processors of the same type or different types (for example, a combination of a plurality of FPGAs or a combination of a CPU and an FPGA). Moreover, a plurality of the processing units may be composed of one processor.

A first example of the configuration in which the plurality of processing units are composed of one processor is a form in which one processor is composed of a combination of one or more CPUs and software and the processor functions as the plurality of processing units, as represented by the computer, such as a client and a server. Second, as represented by a system on chip (SoC) or the like, there is a form of using a processor that realizes the function of the entire system including the plurality of processing units by one integrated circuit (IC) chip. As described above, various processing units are composed of one or more of the various processors as the hardware structure.

Further, more specifically, an electric circuit (circuitry) obtained by combining circuit elements, such as semiconductor elements, can be used as the hardware structure of the various processors.

Moreover, in the embodiments described above, the aspect has been described in which the information processing program 110 is stored (installed) in advance in the non-volatile memory 103, but the disclosed technology is not limited to this. The information processing program 110 may be provided in a form of being recorded in the recording medium, such as a compact disc read only memory (CD-ROM), a digital versatile disc read only memory (DVD-ROM), and a universal serial bus (USB) memory. Moreover, the information processing program 110 may be provided in a form being downloaded from an external device via a network. That is, the program described in the present embodiment (that is, a program product) may be provided by the recording medium or in a form of being distributed from an external computer.

What is claimed is:

1. An information processing apparatus comprising:
at least one processor,
wherein the processor is configured to:
acquire at least one of input information which is input to a user terminal device used by a user, or biological information which is measured for the user, and
derive an examination method for making a diagnosis for a body of the user as a recommended examination method based on at least one of the acquired input information or biological information.

2. The information processing apparatus according to claim 1,
wherein the processor is configured to present the recommended examination method in a case in which an examination order is created for the user.

3. The information processing apparatus according to claim 1,
wherein the processor is configured to
generate auxiliary medical information indicating a physical condition of the user based on at least one of the input information or the biological information,
derive the recommended examination method based on the auxiliary medical information, and
present the recommended examination method together with the auxiliary medical information.

4. The information processing apparatus according to claim 1,
wherein the processor is configured to
receive designation of an examination method to be included in an examination order for the user, and
present, among the examination methods derived as the recommended examination method, an examination method, which matches the examination method designated as the examination method to be included in the examination order and an examination method, which does not match the examination method designated as the examination method to be included in the examination order, in a distinguishable manner.

5. The information processing apparatus according to claim 1,
wherein the processor is configured to include a designated examination method among the examination methods derived as the recommended examination method, in an examination order.

6. The information processing apparatus according to claim 1,
wherein the input information includes a search keyword input to a search engine.

7. The information processing apparatus according to claim 1,
wherein the input information includes a content transmitted or dispatched to a specified or unspecified party.

8. The information processing apparatus according to claim 1,
wherein the processor is configured to
perform processing of selecting input information including a specific word related to a physical condition of the user out of the acquired input information and recording the selected input information in a recording medium, and
derive the recommended examination method based on the input information recorded in the recording medium.

9. The information processing apparatus according to claim 1,
wherein the processor is configured to
acquire medical document data in which medical information obtained by a medical examination of the user is recorded, and
derive the recommended examination method based on the medical information.

10. The information processing apparatus according to claim 9,
wherein the processor is configured to
derive a first recommended examination method based on at least one of the input information or the biological information,
derive a second recommended examination method based on the medical information, and
give information indicating a degree of recommendation to each of an examination method derived as the first recommended examination method and an examination method derived as the second recommended examination method, depending on whether the examination method is derived as any of the first recommended examination method or the second recommended examination method.

11. The information processing apparatus according to claim 1,
wherein the processor is configured to acquire the biological information which is measured by a wearable device worn by the user.

12. An information processing method executed by at least one processor provided in an information processing apparatus, the method comprising:
acquiring at least one of input information which is input to a user terminal device used by a user, or biological information which is measured for the user; and
deriving an examination method for making a diagnosis for a body of the user as a recommended examination method based on at least one of the acquired input information or biological information.

13. A non-transitory computer-readable storage medium storing an information processing program causing at least one processor provided in an information processing apparatus to execute a process comprising:
acquiring at least one of input information which is input to a user terminal device used by a user, or biological information which is measured for the user; and
deriving an examination method for making a diagnosis for a body of the user as a recommended examination method based on at least one of the acquired input information or biological information.
